(19) United States
(12) Patent Application Publication
(10) Pub. No.: US 2024/0173439 A1
(43) Pub. Date: May 30, 2024

(54) FLUORINE-CONTAINING COMPOUND AND CONTRAST MEDIUM

(71) Applicant: TDK CORPORATION, Tokyo (JP)

(72) Inventor: Naoko YANAI, Tokyo (JP)

(73) Assignee: TDK CORPORATION, Tokyo (JP)

(21) Appl. No.: 18/280,627

(22) PCT Filed: Mar. 14, 2022
(86) PCT No.: PCT/JP2022/011250

§ 371 Date: Sep. 6, 2023

(30) Foreign Application Priority Data
Mar. 17, 2021 (JP) 2021-043725
Oct. 29, 2021 (JP) 2021-177507

(51) Int. Cl. A61K 49/10; C07D 211/94

(52) U.S. Cl. CPC A61K 49/10; C07D 211/94

(57) ABSTRACT

A fluorine-containing compound represented by Formula (1) (R¹, R², R³, and R⁴ in Formula (1) each represent a C1-10 alkyl group unsubstituted or substituted with a substituent containing no fluorine atoms, and X is any of Formulae (2-1), (2-2), and (2-3).)

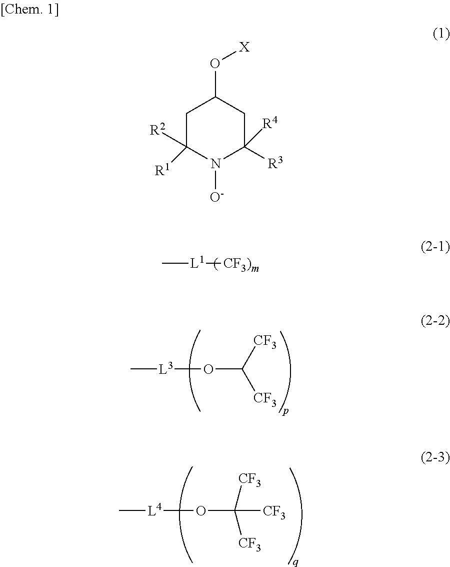

### TECHNICAL FIELD

The present invention relates to a fluorine-containing compound and a contrast medium.

Priority is claimed on Japanese Patent Application No. 2021-043725, filed on Mar. 17, 2021 and Japanese Patent Application No. 2021-177507, filed on Oct. 29, 2021, the contents of which are incorporated herein by reference.

### BACKGROUND ART

Magnetic resonance image (hereinafter sometimes referred to as “MRI”) diagnosis is widely used in the medical field for both basic research and clinical application as one of diagnostic imaging methods along with X-ray diagnosis and ultrasound (US) diagnosis.

Currently, ¹H-MRI using protons (¹H) as detection nuclei is used for medical MRI. ¹H-MRI captures and images the magnetic environment of water molecules present in vivo. A difference occurs in the magnetic environment of protons between lesion tissue and normal tissue in vivo. This appears as a difference in ¹H-MRI and serves as diagnostic information. In addition, water molecules are present almost everywhere in vivo. For this reason. ¹H-MRI can be used for whole-body imaging.

Nuclides detectable by MRI include ¹⁹F, ²³Na, ³¹P, ¹⁵N, ¹³C, and the like in addition to ¹H. MRI using these elements as detection nuclei provides information different from ¹H-MRI.

Among these, MRI using ¹⁹F as a detection nucleus is expected to be used as a next-generation diagnostic method following ¹H-Mm diagnosis. This is because fluorine is an inexpensive element with a natural abundance ratio of 100%, the detection sensitivity of ¹⁹F is as high as 83% of that of ¹H, and the gyromagnetic ratio of ¹⁹F is close to that of a proton, and therefore, imaging can be performed with a conventional ¹H-MRI device.

In addition, ¹⁹F detectable by MRI is almost non-existent in vivo. For this reason, by using a fluorine atom-containing compound in a contrast medium, ¹⁹F-MRI diagnosis using ¹⁹F as a tracer is possible. For example, positional information of lesion portions can be obtained from ¹⁹F-MRI using a fluorine compound, which recognizes and accumulates endogenous changes caused by a disease, in a contrast medium. This method is useful for diagnosing lesion portions that do not cause morphological changes that could not be detected by conventional diagnostic imaging methods.

Currently, there is a nuclear medicine technique as a method of obtaining image information specific to a lesion portion. Nuclear medicine techniques use radiopharmaceuticals in which radioisotopes are used. Specifically, nuclear medicine techniques include a positron emission tomography (PET) examination, and a single photon emission computed tomography (SPECT) examination. However, the nuclear medicine techniques have problems such as large-scale apparatuses for synthesizing radioisotopes and a risk of radiation exposure.

¹⁹F-MRI diagnosis does not cause the above-described problems in nuclear medicine techniques. In addition, in ¹⁹F-MRI diagnosis, by extracting information such as chemical shift, diffusion, and relaxation time, more diagnostic information can be obtained in addition to the positional information of the lesion portions. In addition, by taking ¹⁹F-MRI and ¹H-MRI simultaneously in one diagnosis and superimposing the images, it is possible to obtain useful diagnostic information in which anatomical information and functional information coexist.

Contrast media for MRI diagnosis using fluorine as a detection nucleus are disclosed, for example, in Patent Documents 1 and 2.

Patent Document 1 discloses lactic acid-glycolic acid copolymer (PLGA) particles containing perfluoro crown ether and gadolinium complexes. In addition, Patent Document 2 discloses a fluorine-containing porphyrin complex and a contrast medium compound which can be used in MRI using fluorine as a detection nucleus.

However, since the contrast media disclosed in Patent Documents 1 and 2 contain metal ions, there is a concern about their safety in vivo.

In addition, Patent Document 3 discloses a compound having a nitroxide covalently bound to a fluorine-containing compound. However, since the fluorine-containing compound disclosed in Patent Document 3 is easily reduced by a reducing agent such as ascorbic acid (for example, refer to Non-Patent Document 1), it has a problem of stability in vivo.

### CITATION LIST

### Patent Document

### Patent Document 1

Published Japanese Translation No. 2015-534549 of the PCT International Publication

### Patent Document 2

Japanese Unexamined Patent Application, First Publication No. H11-217385

### Patent Document 3

U.S. Pat. No. 5,362,477(B)

### Non-Patent Document

### Non-Patent Document 1

ACS Central Science, 2017, 3, 800-811

### SUMMARY OF INVENTION

### Technical Problem

The conventional contrast media for MRI diagnosis using fluorine as a detection nucleus do not provide high-sensitivity MRI and are not highly stable in vivo.

The present invention has been made in consideration of the above-described circumstances, and an object of the invention is to provide a fluorine-containing compound which is used as a material for a contrast medium for magnetic resonance image diagnosis using fluorine as a detection nucleus and is highly stable in vivo and from which a high-sensitivity magnetic resonance image is obtained.

In addition, another object of the present invention is to provide a contrast medium for magnetic resonance image diagnosis using fluorine as a detection nucleus which contains the fluorine-containing compound of the present invention and is highly stable in vivo and from which a high-sensitivity image can be obtained.

### Solution to Problem

[1] A fluorine-containing compound represented by General Formula (1) below.

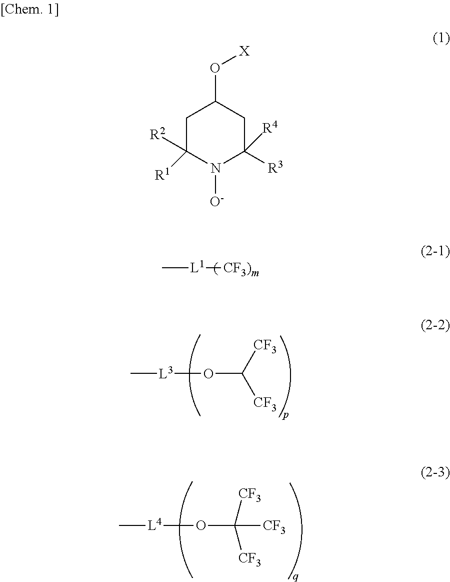

(In General Formula (1), R¹, R², R³, and R⁴ each independently represent a C1-10 alkyl group unsubstituted or substituted with a substituent containing no fluorine atoms, and X is a substituent represented by any of General Formulae (2-1), (2-2), and (2-3).)

(In General Formula (2-1), L¹ is either a C1-16 chain hydrocarbon group unsubstituted or substituted with a substituent containing no fluorine atoms or a linking group containing a C6-12 aryl group unsubstituted or substituted with a substituent containing no fluorine atoms, and m is an integer of 1 to 5.)

(In General Formula (2-2), L³ is either a C1-16 chain hydrocarbon group unsubstituted or substituted with a substituent containing no fluorine atoms or a linking group containing a C6-12 aryl group unsubstituted or substituted with a substituent containing no fluorine atoms, and p is an integer of 1 to 5.)

(In General Formula (2-3), L⁴ is either a C1-16 chain hydrocarbon group unsubstituted or substituted with a substituent containing no fluorine atoms or a linking group containing a C6-12 aryl group unsubstituted or substituted with a substituent containing no fluorine atoms, and q is an integer of 1 to 5.)

[2] The fluorine-containing compound according to [1], in which R¹, R², R³, and R⁴ in General Formula (1) above each independently represent a C1-5 alkyl group unsubstituted or substituted with a substituent containing no fluorine atoms.

[3] The fluorine-containing compound according to [1] or [2], in which L¹ in General Formula (2-1), L³ in General Formula (2-2), and L⁴ in General Formula (2-3) are C1-10 chain hydrocarbon groups unsubstituted or substituted with a substituent containing no fluorine atoms.

[4] The fluorine-containing compound according to [1] or [2], in which L¹ in General Formula (2-1), L³ in General Formula (2-2), and L⁴ in General Formula (2-3) are linking groups containing a phenyl group.

[5] The fluorine-containing compound according to any one of [1] to [4], in which m in General Formula (2-1) is an integer of 1 to 3, and p in General Formula (2-2) and q in General Formula (2-3) are each 1 or 2.

[6] The fluorine-containing compound according to any one of [1] to [5], which is used in a contrast medium for magnetic resonance image diagnosis using fluorine as a detection nucleus.

[7] A contrast medium for magnetic resonance image diagnosis using fluorine as a detection nucleus, including: the fluorine-containing compound according to any one of [1] to [6].

### Advantageous Effects of Invention

The fluorine-containing compound of the present invention is a compound represented by General Formula (1) above. For this reason, it is highly stable in vivo. In addition, the fluorine-containing compound of the present invention is used as a material for a contrast medium for magnetic resonance image diagnosis using fluorine as a detection nucleus to obtain a high-sensitivity magnetic resonance image.

A contrast medium of the present invention contains the fluorine-containing compound of the present invention. For this reason, the contrast medium of the present invention is highly stable in vivo. In addition, the contrast medium of the present invention is used as a contrast medium for magnetic resonance image diagnosis using fluorine as a detection nucleus to obtain a high-sensitivity magnetic resonance image.

### BRIEF DESCRIPTION OF DRAWINGS

FIG. 1 is a ¹⁹F spin-lattice relaxation time (T1)-weighted ¹⁹F-MRI image for Example 1 (compound 11).

FIG. 2 is a ¹⁹F spin-lattice relaxation time (T1)-weighted ¹⁹F-MRI image for Comparative Example 1 (compound A1).

### DESCRIPTION OF EMBODIMENT

Hereinafter, a fluorine-containing compound and a contrast medium of the present invention will be described in detail.

### [Fluorine-Containing Compound]

A fluorine-containing compound of the present embodiment is represented by General Formula (1) below.

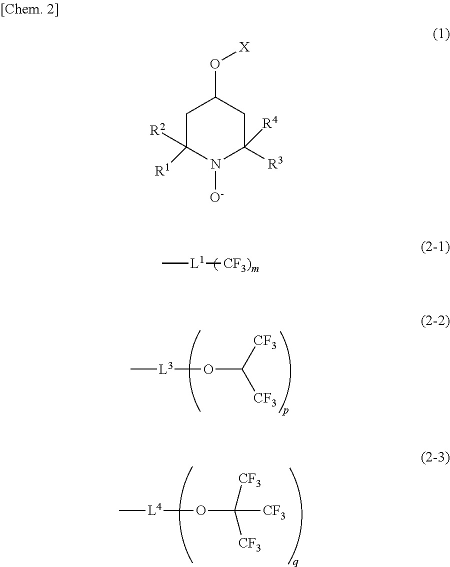

(In General Formula (1), R¹, R², R³, and R⁴ each independently represent a C1-10 alkyl group unsubstituted or substituted with a substituent containing no fluorine atoms, and X is a substituent represented by any of General Formulae (2-1), (2-2), and (2-3).)

(In General Formula (2-1), L¹ is either a C1-16 chain hydrocarbon group unsubstituted or substituted with a substituent containing no fluorine atoms or a linking group containing a C6-12 aryl group unsubstituted or substituted with a substituent containing no fluorine atoms, and m is an integer of 1 to 5.)

(In General Formula (2-2), L³ is either a C1-16 chain hydrocarbon group unsubstituted or substituted with a substituent containing no fluorine atoms or a linking group containing a C6-12 aryl group unsubstituted or substituted with a substituent containing no fluorine atoms, and p is an integer of 1 to 5.)

(In General Formula (2-3), L⁴ is either a C1-16 chain hydrocarbon group unsubstituted or substituted with a substituent containing no fluorine atoms or a linking group containing a C6-12 aryl group unsubstituted or substituted with a substituent containing no fluorine atoms, and q is an integer of 1 to 5.)

Here, the reason why the contrast medium containing the fluorine-containing compound of the present embodiment is highly stable in vivo and a high-sensitivity magnetic resonance image (MRI) can be obtained when the contrast medium is used as a contrast medium for MRI diagnosis using fluorine as a detection nucleus will be described.

In order to obtain high-sensitivity ¹⁹F-MRI, it is preferable to use a fluorine-containing compound with a short ¹⁹F spin-lattice relaxation time (T1) as a fluorine-containing compound contained in a contrast medium. The shorter the T1 of the fluorine-containing compound, the shorter the repetition time can be set. For this reason, the amount of signal obtained per unit time is increased, and a high-sensitivity image can be obtained. On the other hand, if the ¹⁹F spin-spin relaxation time (T2) of the fluorine-containing compound is too short, the signal intensity will decrease.

The ¹⁹F spin-lattice relaxation time (T1) and ¹⁹F spin-spin relaxation time (T2) of a fluorine-containing compound are affected by a paramagnetic relaxation enhancement (PRE) effect. The PRE effect is a phenomenon in which T1 and T2 of MRI observation nuclei in the vicinity of unpaired electron spins possessed by a paramagnetic material are shortened by the unpaired electron spins.

The PRE effect is inversely proportional to the sixth power of the distance between a paramagnetic substance and a MRI observation nucleus (a fluorine atom in the present embodiment) relaxed by the paramagnetic material. Accordingly, in the fluorine-containing compound represented by Formula (1) of the present embodiment, the shorter the distance between the fluorine atom and a nitroxide radical which is a paramagnetic material, the shorter T1 and T2 are. In the fluorine-containing compound represented by Formula (1), a substituent (X in Formula (1)) to which the fluorine atom is bound at a terminal is bound to carbon at the 4-position of the piperidine ring through an oxygen atom. For this reason, the distance between the nitroxide radical and the fluorine atom is appropriate, T1 is sufficiently short, and T2 can be sufficiently secured. Accordingly, the fluorine-containing compound represented by Formula (1) is used in a contrast medium for MRI diagnosis using fluorine as a detection nucleus to obtain a high-sensitivity magnetic resonance image.

In addition, unlike closed-shell species, an organic radical has a semi-occupied molecular orbital (SOMO) with unpaired electrons between the highest occupied molecular orbital (HOMO) and the lowest unoccupied molecular orbital (LUMO). A redox process of organic radicals corresponds to an electron transfer process in a SOMO. The reduction reaction of organic radicals due to a reducing agent such as ascorbic acid is more likely to occur when the energy difference between the HOMO of the reducing agent and the SOMO of the organic radicals is smaller. Accordingly, the lower the SOMO energy level of the organic radicals, the easier they are to reduce.

In the fluorine-containing compound represented by Formula (1) of the present embodiment, three carbon atoms are arranged between a nitrogen atom of the piperidine ring and the substituent represented by X in Formula (1), and two or more carbon atoms are arranged between the fluorine atom and the oxygen atom to which the substituent represented by X is bound. Accordingly, in the fluorine-containing compound represented by Formula (1), the nitroxide radical and the fluorine atom are arranged at sufficiently distant positions, and therefore the nitroxide radical is less susceptible to electronic influence from the fluorine atom. Accordingly, in the fluorine-containing compound represented by Formula (1), the SOMO energy level of the nitroxide radical does not decrease due to the fluorine atom which is an electron withdrawing group. Accordingly, the SOMO of the nitroxide radical in the fluorine-containing compound of the present embodiment has a sufficiently large energy difference from the HOMO of the reducing agent such as ascorbic acid. Accordingly, the fluorine-containing compound represented by Formula (1) is less likely to be reduced in vivo and highly stable in vivo.

Moreover, since the fluorine-containing compound represented by Formula (1) of the present embodiment is a non-metal compound containing no metal, it is highly safer in vivo compared with a contrast medium containing metal ions. Accordingly, the fluorine-containing compound of the present embodiment is suitable as a material for a contrast medium for magnetic resonance image diagnosis using fluorine as a detection nucleus.

In addition, the fluorine-containing compound represented by Formula (1) of the present embodiment is a compound having a piperidine ring and has a structure similar to 4-hydroxy-2,2,6,6-tetramethylpiperidine-1-oxyl free radical (TEMPOL) which is highly safe in vivo. For this reason, it is inferred that the fluorine-containing compound represented by Formula (1) of the present embodiment would have a higher in vivo stability than, for example, a fluorine-containing compound containing a pyrrolidine ring.

R¹, R², R³, and R⁴ in the fluorine-containing compound represented by Formula (1) of the present embodiment are each independently a C1-10 alkyl group unsubstituted or substituted with a substituent containing no fluorine atoms and preferably a C1-5 alkyl group unsubstituted or substituted with a substituent containing no fluorine atoms. Since R¹, R², R³, and R⁴ are each independently a substituted or unsubstituted C1-10 alkyl group, the fluorine-containing compound represented by Formula (1) is easily synthesized. In addition, if R¹, R², R³, and R⁴ are substituted or unsubstituted C2-10 alkyl groups, they become appropriately bulky and can prevent the approach of a reducing agent to the nitroxide radical. If the number of carbon atoms in the above-described alkyl group is 5 or less, synthesis of the fluorine-containing compound represented by Formula (1) becomes much easier, which is preferable.

In a case where R¹, R², R³, and R⁴ contained in the fluorine-containing compound represented by Formula (1) have a substituent containing no fluorine atoms, a methyl group or an ethyl group can be used as the substituent, for example.

Specifically, R¹, R², R³, and R⁴ in the fluorine-containing compound represented by Formula (1) of the present embodiment are preferably a methyl group or an ethyl group, and more preferably a methyl group for easy synthesis.

In the fluorine-containing compound represented by Formula (1) of the present embodiment, X represents a substituent represented by any of Formulae (2-1), (2-2), and (2-3). For this reason, in the fluorine-containing compound represented by Formula (1), the distance between the nitroxide radical and the fluorine atom is appropriate, T1 is sufficiently short, and T2 can be sufficiently secured. Accordingly, the fluorine-containing compound represented by Formula (1) is used in a contrast medium for MRI diagnosis using fluorine as a detection nucleus to obtain a high-sensitivity image. In addition, since the distance between the nitroxide radical and the fluorine atom is appropriate, the nitroxide radical is less susceptible to electronic influence from the fluorine atom. Moreover, since the substituents represented by Formulae (2-1), (2-2), and (2-3) are all bulky, the approach of a reducing agent to the nitroxide radical is three-dimensionally blocked and prevented. Accordingly, the fluorine-containing compound represented by Formula (1) is less likely to be reduced in vivo and highly stable in vivo.

In the substituent represented by Formula (2-1) contained in the fluorine-containing compound represented by Formula (1), L¹ is either a C1-16 chain hydrocarbon group unsubstituted or substituted with a substituent containing no fluorine atoms or a linking group containing a C6-12 aryl group unsubstituted or substituted with a substituent containing no fluorine atoms.

If L¹ is a C1-16 chain hydrocarbon group unsubstituted or substituted with a substituent containing no fluorine atoms, the distance between the nitroxide radical and the fluorine atom is appropriate. In the case where L¹ is a C1-16 chain hydrocarbon group unsubstituted or substituted with a substituent containing no fluorine atoms, a C1-10 chain hydrocarbon group unsubstituted or substituted with a substituent containing no fluorine atoms is preferable, and a C1-5 chain hydrocarbon group is more preferable. If the above-described chain hydrocarbon group has 16 or less carbon atoms, the distance between the nitroxide radical and the fluorine atom does not become too long, and T1 is sufficiently short. If the above-described chain hydrocarbon group has 10 or less carbon atoms, T1 becomes shorter, which is preferable.

In a case where the C1-16 chain hydrocarbon group which is unsubstituted or substituted with a substituent containing no fluorine atoms and represented by L¹ has a substituent, a substituent, such as a methyl group, an ethyl group, or a phenyl group, containing no fluorine atoms can be used.

In the case where L¹ is the C1-16 chain hydrocarbon group unsubstituted or substituted with a substituent containing no fluorine atoms, any one selected from —CH₂—, —(CH₂)₂—, —(CH₂)₃—, and —(CH₂)₄— is more preferable, and any one selected from —(CH₂)₂—. —(CH₂)₃—, and —(CH₂)₄— is particularly preferable. In this case, the distance between the nitroxide radical and the fluorine atom is more appropriate. As a result, a fluorine-containing compound in which the nitroxide radical is less susceptible to electronic influence from the fluorine atom and which has a high in vivo stability is obtained. Moreover, since this fluorine-containing compound has a shorter T1, in a case where this is used as a contrast medium for MRI diagnosis using fluorine as a detection nucleus, a higher-sensitivity image is obtained.

If L¹ is a linking group containing a C6-12 aryl group unsubstituted or substituted with a substituent containing no fluorine atoms, the distance between the nitroxide radical and the fluorine atom is appropriate. In the case where L¹ is a linking group containing a C6-12 aryl group unsubstituted or substituted with a substituent containing no fluorine atoms, a linking group containing a phenyl group is preferable. If the above-described aryl group has 12 or less carbon atoms, the distance between the nitroxide radical and the fluorine atom does not become too long, and T1 is sufficiently short. In the case where L¹ is a linking group containing a phenyl group, the fluorine-containing compound represented by Formula (1) is easily synthesized, which is preferable.

In a case where the linking group which contains a C6-12 aryl group unsubstituted or substituted with a substituent containing no fluorine atoms and represented by L¹ has a substituent, a substituent, such as a p-phenylene group, m-phenylene group, o-phenylene group, a biphenylene group, or a benzylene group, containing no fluorine atoms can be used.

In the case where L¹ is a linking group containing a C6-12 aryl group unsubstituted or substituted with a substituent containing no fluorine atoms, any one selected from a p-phenylene group, a m-phenylene group, and an o-phenylene group is preferable. In this case, the distance between the nitroxide radical and the fluorine atom is more appropriate, and L¹ is bulky. As a result, a fluorine-containing compound in which the nitroxide radical is less susceptible to electronic influence from the fluorine atom and which is highly stabler in vivo is obtained. Furthermore, since this fluorine-containing compound has a sufficiently short T1, in a case where this is used as a contrast medium for MRI diagnosis using fluorine as a detection nucleus, a higher-sensitivity image is obtained.

In the substituent represented b Formula (2-1) contained in the fluorine-containing compound represented by Formula (1), m is an integer of 1 to 5.

In the substituent represented by Formula (2-1), in the case where L¹ is a C1-16 chain hydrocarbon group unsubstituted or substituted with a substituent containing no fluorine atoms, m is preferably an integer of 1 to 3. In a case where L¹ is a C1-16 chain hydrocarbon group unsubstituted or substituted with a substituent containing no fluorine atoms and the fluorine-containing compound in which m is an integer of 1 to 3 is used as a contrast medium for magnetic resonance image diagnosis using fluorine as a detection nucleus, a single ¹⁹F-MRI peak is exhibited. For this reason, high-quality ¹⁹F-MRI in which chemical shift artifacts are suppressed is obtained. In addition, a fluorine-containing compound in which m is 3 has a larger number of fluorine atoms exhibiting a single ¹⁹F-MRI peak than a fluorine-containing compound in which m is 1 or 2, and a strong signal intensity is obtained, which is preferable.

In the case where L¹ is a linking group containing a C6-12 aryl group unsubstituted or substituted with a substituent containing no fluorine atoms, m is an integer of 1 to 5 and is preferably 1 or 2. In the case where a fluorine-containing compound in which L¹ is a linking group containing a C6-12 aryl group unsubstituted or substituted with a substituent containing no fluorine atoms and m is 1 or 2 is used as a contrast medium for magnetic resonance image diagnosis using fluorine as a detection nucleus, a single ¹⁹F-MRI peak is exhibited. For this reason, high-quality ¹⁹F-MRI in which chemical shift artifacts are suppressed is obtained. In addition, a fluorine-containing compound in which m is 2 has a larger number of fluorine atoms exhibiting a single ¹⁹F-MRI peak than a fluorine-containing compound in which m is 1, and a stronger signal intensity is obtained.

Similarly to L¹ in Formula (2-1), L³ in Formula (2-2) and L⁴ in General Formula (2-3) contained in the fluorine-containing compound represented by Formula (1) are each independently either a C1-16 chain hydrocarbon group unsubstituted or substituted with a substituent containing no fluorine atoms or a linking group containing a C6-12 aryl group unsubstituted or substituted with a substituent containing no fluorine atoms.

In a case where L³ in Formula (2-2) and L⁴ in General Formula (2-3) are a C1-16 chain hydrocarbon group unsubstituted or substituted with a substituent containing no fluorine atoms similarly to L¹ in Formula (2-1), these are preferably a C1-10 chain hydrocarbon group unsubstituted or substituted with a substituent containing no fluorine atoms, more preferably a C1-5 chain hydrocarbon group, and still more preferably any one selected from —CH₂—, —(CH₂)₂—, —(CH₂)₃—, and —(CH₂)₄—.

In a case where L³ in Formula (2-2) and L⁴ in General Formula (2-3) are a linking group containing a C6-12 aryl group unsubstituted or substituted with a substituent containing no fluorine atoms similarly to L¹ in Formula (2-1), these are preferably a linking group containing a phenyl group and more preferably any one selected from a p-phenylene group, a m-phenylene group, and an o-phenylene group.

p in Formula (2-2) and q in Formula (2-3) contained in the fluorine-containing compound represented by Formula (1) are each independently an integer of 1 to 5, and are preferably 1 or 2 and most preferably 1 for each synthesis. In a case where a fluorine-containing compound in which p and q are each independently 1 or 2 is used as a contrast medium for magnetic resonance image diagnosis using fluorine as a detection nucleus, a single ¹⁹F-MRI peak is exhibited. For this reason, high-quality ¹⁹F-MRI in which chemical shift artifacts are suppressed is obtained. In addition, a fluorine-containing compound in which p and q are 2 has a larger number of fluorine atoms exhibiting a single ¹⁹F-MRI peak than a fluorine-containing compound in which p and q are 1, and a stronger signal intensity is obtained.

Specifically, the fluorine-containing compound represented by Formula (1) is preferably any one of fluorine-containing compounds represented by Formulae (11) to (29) below.

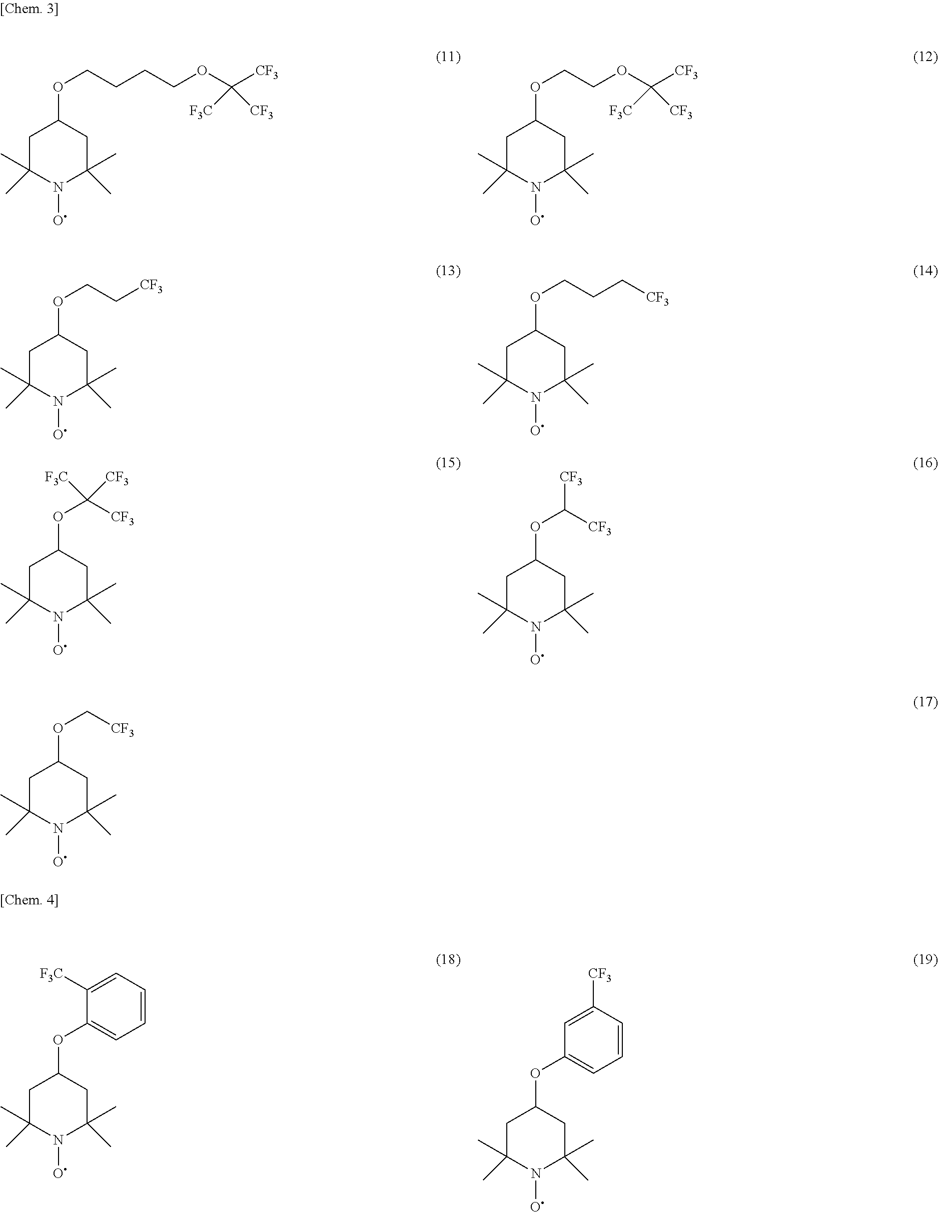

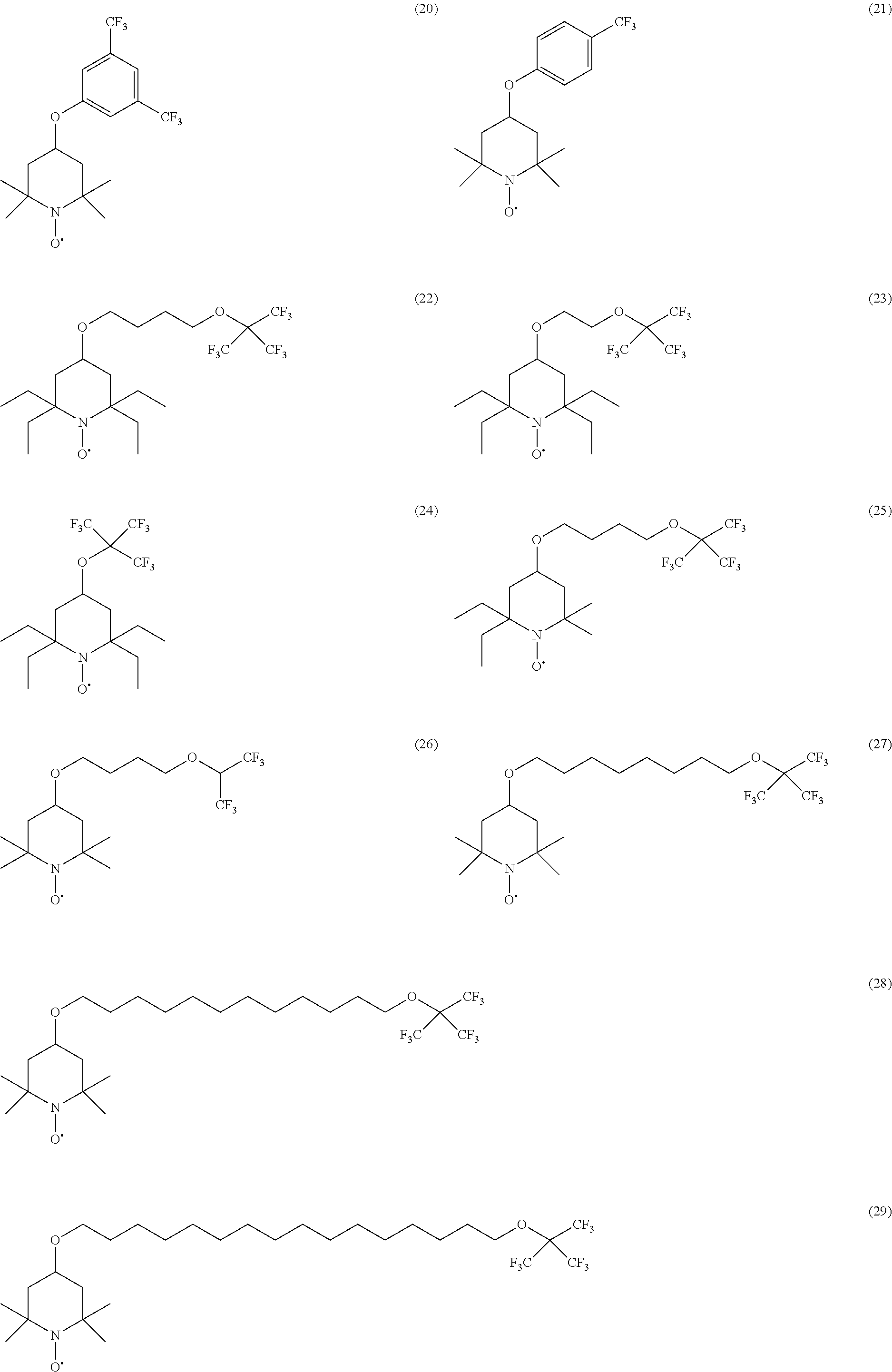

### [Method for Producing Fluorine-Containing Compound]

Next, a method for producing a fluorine-containing compound of the present embodiment represented by Formula (1) will be described as an example.

The method for producing a fluorine-containing compound of the present embodiment is not particularly limited, and the fluorine-containing compound can be produced using a well-known conventional production method.

The fluorine-containing compound of the present embodiment represented by Formula (1) can be produced using, for example, a production method shown below.

First, 4-piperidone in which R¹, R², R³, and R⁴ in the fluorine-containing compound represented by Formula (1) are bound to the 2- and 6-positions of the piperidine ring is prepared. Then, this compound is reacted with di-tert-butyldicarbonate to bind a tertiary butoxycarbonyl group (t-Boc group) which is a protecting group to a nitrogen atom of the piperidine ring, thereby obtaining a first intermediate compound. Next, the first intermediate compound is reduced using sodium borohydride to obtain a second intermediate compound in which a hydroxyl group is bound to the 4-position of the piperidine ring.

Next, a compound having a group corresponding to X in the fluorine-containing compound represented by Formula (1) is reacted with the second intermediate compound to obtain a third intermediate compound in which the group corresponding to X is bound to an oxygen atom at the 4-position of the piperidine ring. Thereafter, using dichloromethane and trifluoroacetic acid, the tertiary butoxycarbonyl group which is a protecting group is removed from the nitrogen atom forming the piperidine ring of the third intermediate compound to perform conversion into a nitroxide radical.

The fluorine-containing compound represented by Formula (1) is obtained through the above-described method.

### “Contrast Medium”

A contrast medium of the present embodiment contains a fluorine-containing compound of the present embodiment. The contrast medium of the present embodiment is a contrast medium for magnetic resonance image diagnosis using fluorine as a detection nucleus.

The contrast medium of the present embodiment can be produced, for example, through a method for formulating the fluorine-containing compound of the present embodiment into forms such as a solid formulation, a powder preparation, and a liquid formulation using a well-known formulation technique.

The contrast medium of the present embodiment may contain one kind or two or more kinds of additives, such as an excipient, a stabilizer, a surfactant, a buffer agent, and an electrolyte, used in well-known formulations as necessary in addition to the fluorine-containing compound of the present embodiment.

Since the contrast medium of the present embodiment contains the fluorine-containing compound of the present invention, it is highly stable in vivo. In addition, the contrast medium of the present embodiment is used as a contrast medium for magnetic resonance image diagnosis using fluorine as a detection nucleus to obtain a high-sensitivity magnetic resonance image.

The embodiment of the present invention has been described. However, each configuration and combination thereof in each embodiment are merely an example, and addition, omission, replacement, and other modifications of the configuration can be made within the scope not departing from the gist of the present invention.

### EXAMPLES

### Example 1

### Synthesis of Compound 11

### <Synthesis of tert-butyl-2,2,6,6-tetramethyl-4-oxopiperidine-1-carboxylic acid ester (1-1)

7.762 g (50.0 mmol) of 2,2,6,6-tetramethylpiperidine-4-one was dissolved in 50 mL of tetrahydrofuran (THF) and cooled in an ice bath. 50 mL of a tetrahydrofuran solution of 14.6 mL (105 mmol) of triethylamine (Et₃N) and 7.364 g (52.5 mmol) of di-tert-butyldicarbonate (Boc₂O) was added thereto and stirred at room temperature for 2 hours to cause a reaction.

The reaction solution was concentrated under reduced pressure, and hexane was added thereto. After the resulting solid was filtered off, it was washed with hexane to obtain tert-butyl-2,2,6,6-tetramethyl-4-oxopiperidine-1-carboxylic acid ester (yield of g, percent yield of 90%) represented by Formula (1-1) below which was a target product in which a tertiary butoxycarbonyl group which was a protecting group was bound to a nitrogen atom of a piperidine ring.

### Synthesis of tert-butyl-4-hydroxy-2,2,6,6-tetramethyl-piperidine-1-carboxylic acid ester (1-2)

In an argon stream, 11.491 g (45.0 mmol) of tert-butyl-2,2,6,6-tetramethyl-4-oxopiperidine-1-carboxylic acid ester (1-1) synthesized through the above-described reaction was dissolved in 30 mL of ethanol (EtOH) and cooled in an ice bath. 0.875 g (23.0 mmol) of sodium borohydride was slowly added thereto and stirred at room temperature for 6 hours to cause a reaction.

Saturated saline was added to the reaction solution, extraction was performed with ethyl acetate, and the extract was dried with magnesium sulfate. The extract was concentrated under reduced pressure, and tert-butyl-4-hydroxy-2,2,6,6-tetramethyl-piperidine-1-carboxylic acid ester which was a target product and represented by Formula (1-2) was obtained (yield of 9.844 g, percent yield of 85%).

### Synthesis of Compound (1-3)

In an argon stream, 10 mL of tetrahydrofuran (THF) was added to 2.007 g (46.0 mmol) of 55% sodium hydride and cooled in an ice bath. 50 mL of a tetrahydrofuran solution of 9.844 g (38.3 mmol) of tert-butyl-4-hydroxy-2,2,6,6-tetramethyl-piperidine-1-carboxylic acid ester (1-2) synthesized through the above-described reaction was added thereto over 20 minutes and stirred for 30 minutes to cause a reaction.

30 mL of a tetrahydrofuran solution of 17.018 g (46.0 mmol) of 1-bromo-4-((1,1,1,3,3,3-hexafluoro-2-(trifluoromethyl)propane-2-yl)oxy)butane was added to the reaction solution over 10 minutes and stirred at room temperature for 12 hours to cause a reaction. Water was added to the reaction solution, extraction was performed with diethyl ether, and the extract was dried with magnesium sulfate. The extract was concentrated under reduced pressure, the resulting crude product was purified through silica gel column chromatography (hexane:ethyl acetate=9:1 to 4:1) to obtain a compound which was a target product and represented by Formula (1-3) (yield of 10.485 g, percent yield of 50%).

### Synthesis of 4-(4-((1,1,1,3,3,3-hexafluoro-2-(trifluoromethyl)propane-2-yl)oxy)butoxy)-2,2,6,6-tetra methylpiperidine (1-4)

10.485 g (19.2 mmol) of the compound (1-3) synthesized through the above-described reaction was dissolved in 60 mL of dichloromethane, 11.5 mL (150 mmol) of trifluoroacetic acid (TFA) was added thereto and stiffed at room temperature for 18 hours to cause a reaction, and a tertiary butoxycarbonyl group which was a protecting group was removed. After the reaction solution was concentrated under reduced pressure, water was added thereto, and the organic layer was neutralized with a sodium hydrogen carbonate aqueous solution. After extraction was performed with diethyl ether, the organic layer was washed with saturated saline and dried with magnesium sulfate. The organic layer was concentrated under reduced pressure, and 4-(4-((1,1,1,3,3,3-hexafluoro-2-(trifluoromethyl)propane-2-yl)oxy)butoxy)-2,2,6,6-tetra methylpiperidine which was a target product and represented by Formula (1-4) was obtained (yield of 7.989 g, percent yield of 93%).

### Synthesis of 4-(4-((1,1,1,3,3,3-hexafluoro-2-(trifluoromethyl)propane-2-yl)oxy)butoxy)-2,2,6,6-tetra methylpiperidine-1-oxyl (11)

7.989 g (17.9 mmol) of 4-(4-((1,1,1,3,3,3-hexafluoro-2-(trifluoromethyl)propane-2-yl)oxy)butoxy)-2,2,6,6-tetra methylpiperidine (1-4) synthesized through the above-described reaction, 0.660 g (2.00 mmol) of sodium tungstate dihydrate, and 5 mL of ethanol (EtOH) were mixed with each other and cooled in an ice bath. 15 mL (143 mmol) of a 30% hydrogen peroxide solution was slowly added thereto and stirred at room temperature for 24 hours. Potassium carbonate was added the reaction solution, extraction was performed with chloroform, and the extract was dried with magnesium sulfate.

After the extract was concentrated under reduced pressure, the resulting crude product was purified through silica gel column chromatography (hexane:ethyl acetate=9:1) to obtain 4-(4-((1,1,1,3,3,3-hexafluoro-2-(trifluoromethyl)propane-2-yl)oxy)butoxy)-2,2,6,6-tetra methylpiperidine-1-oxyl (11) which was a target product and represented by Formula (11) (yield of 5.046 g, percent yield of 61%).

When the resulting compound was subjected to mass spectrometry, a peak was confirmed at m/z=462 (M⁺). From this, it was confirmed that the synthesized compound is a compound represented by Formula (11). In addition, the purity of the compound represented by Formula (11) which was conformed through high-performance liquid chromatography (HPLC) was 97.3%.

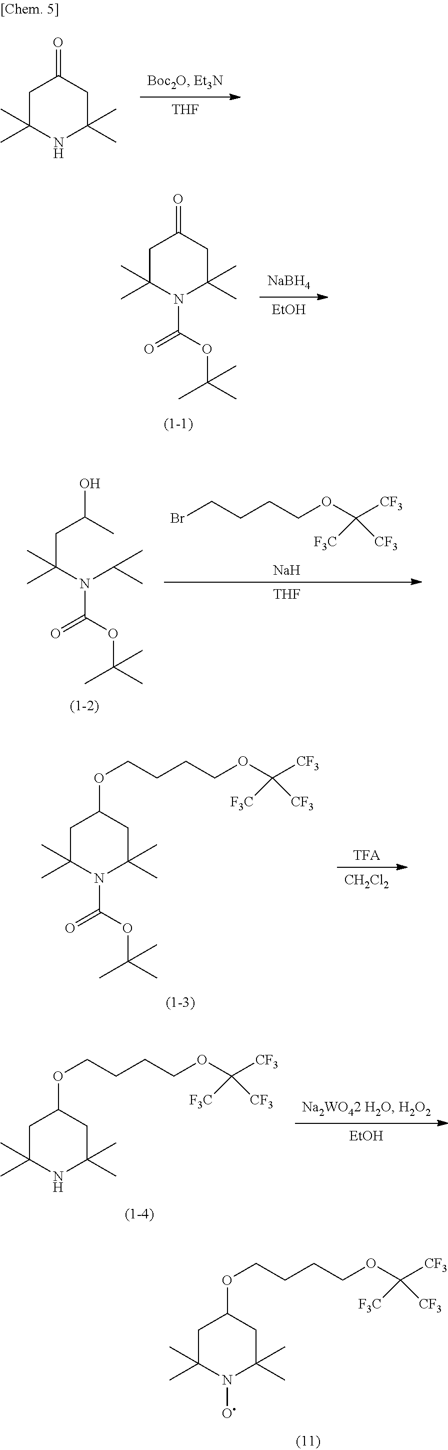

### Example 2

### Synthesis of Compound 12

A compound represented by Formula (12) which was a target product was synthesized in the same manner as in Example 1 except that 2-(2-bromoethoxy)-1,1,1,3,3,3-hexafluoro-2-(trifluoromethyl)propane was used instead of 1-bromo-4-((1,1,1,3,3,3-hexafluoro-2-(trifluoromethyl)propane-2-yl)oxy)butane.

When the resulting compound was subjected to mass spectrometry, a peak was confirmed at m/z=434 (M⁺). From this, it was confirmed that the synthesized compound is a compound represented by Formula (12). In addition, the purity of the compound represented by Formula (12) which was conformed through high-performance liquid chromatography (HPLC) was 97.0%.

### Example 3

### Synthesis of Compound 13

A compound represented by Formula (13) which was a target product was synthesized in the same manner as in Example 1 except that 3,3,3-trifluoro-1-iodopropane was used instead of 1-bromo-4-((1,1,1,3,3,3-hexafluoro-2-(trifluoromethyl)propane-2-yl)oxy)butane.

When the resulting compound was subjected to mass spectrometry, a peak was confirmed at m/z=268 (M⁺). From this, it was confirmed that the synthesized compound is a compound represented by Formula (13). In addition, the purity of the compound represented by Formula (13) which was conformed through high-performance liquid chromatography (HPLC) was 96.5%.

### Example 4

### Synthesis of Compound 14

A compound represented by Formula (14) which was a target product was synthesized in the same manner as in Example 1 except that 4,4,4-trifluoro-1-iodobutane was used instead of 1-bromo-4-((1,1,1,3,3,3-hexafluoro-2-(trifluoromethyl)propane-2-yl)oxy)butane.

When the resulting compound was subjected to mass spectrometry, a peak was confirmed at m/z=282 (M⁺). From this, it was confirmed that the synthesized compound is a compound represented by Formula (14). In addition, the purity of the compound represented by Formula (14) which was conformed through high-performance liquid chromatography (HPLC) was 96.9%.

### Example 5

### Synthesis of Compound 15

### Synthesis of Compound (1-5)

In an argon stream, 2.572 g (10.0 mmol) of tert-butyl-4-hydroxy-2,2,6,6-tetramethyl-piperidine-1-carboxylic acid ester (1-2) synthesized through the above-described reaction, 2.08 mL (15.0 mmol) of nonafluoro-tert-butanol, 3.934 g (15.0 mmol) of triphenylphosphine (PPh₃), and 40 mL of tetrahydrofuran (THF) were mixed with each other and cooled in an ice bath. 2.92 mL (15.0 mmol) of diisopropyl azodicarboxylate (iPrO₂CNNCO₂iPr) was added dropwise thereto over 10 minutes and stiffed at room temperature for 24 hours to cause a reaction.

The reaction solution was concentrated under reduced pressure and purified through silica gel column chromatography (hexane:ethyl acetate=9:1 to 4:1) to obtain a compound which was a target product and represented by Formula (1-5) (yield of 2.471 g, percent yield of 52%).

### Synthesis of 4-((1,1,1,3,3,3-hexafluoro-2-(trifluoromethyl)propane-2-yl)oxy)-2,2,6,6-tetramethylpiper idine (1-6)

2.471 g (5.20 mmol) of the compound represented by Formula (1-5) synthesized through the above-described reaction was dissolved in 15 mL of dichloromethane, 3.1 mL (40.0 mmol) of trifluoroacetic acid (TFA) was added thereto and stirred at room temperature for 18 hours to cause a reaction, and a tertiary butoxycarbonyl group which was a protecting group was removed. After the reaction solution was concentrated under reduced pressure, water was added thereto, and the organic laver was neutralized with a sodium hydrogen carbonate aqueous solution. After extraction was performed with diethyl ether, the organic layer was washed with saturated saline and dried with magnesium sulfate. The organic layer was concentrated under reduced pressure, and 4-((1,1,1,3,3,3-hexafluoro-2-(trifluoromethyl)propane-2-yl)oxy)-2,2,6,6-tetramethylpiperid idine which was a target product and represented by Formula (1-6) was obtained (yield of 1.853 g, percent yield of 95%).

### Synthesis of 4-((1,1,1,3,3,3-hexafluoro-2-(trifluoromethyl)propane-2-yl)oxy)-2,2,6,6-tetramethylpiper idine-1-oxyl(15)

1.853 g (4.94 mmol) of 4-((1,1,1,3,3,3-hexafluoro-2-(trifluoromethyl)propane-2-yl)oxy)-2,2,6,6-tetramethylpiper idine (1-6) synthesized through the above-described reaction, 0.165 g (0.50 mmol) of sodium tungstate dihydrate, and 5 mL of ethanol (EtOH) were mixed with each other and cooled in an ice bath. 15 mL (143 mmol) of a 30% hydrogen peroxide solution was slowly added thereto and stirred at room temperature for 24 hours to cause a reaction. Potassium carbonate was added the reaction solution, extraction was performed with chloroform, and the extract was dried with magnesium sulfate.

After the extract was concentrated under reduced pressure, the resulting crude product was purified through silica gel column chromatography (hexane:ethyl acetate=9:1) to obtain 4-((1,1,1,3,3,3-hexafluoro-2-(trifluoromethyl)propane-2-yl)oxy)-2,2,6,6-tetramethylpiper idine-1-oxyl which was a target product and represented by Formula (15) (yield of 1.118 g, percent yield of 58%).

When the resulting compound was subjected to mass spectrometry, a peak was confirmed at m/z=390 (M⁺). From this, it was confirmed that the synthesized compound is a compound represented by Formula (15). In addition, the purity of the compound represented by Formula (15) which was conformed through high-performance liquid chromatography (HPLC) was 95.6%.

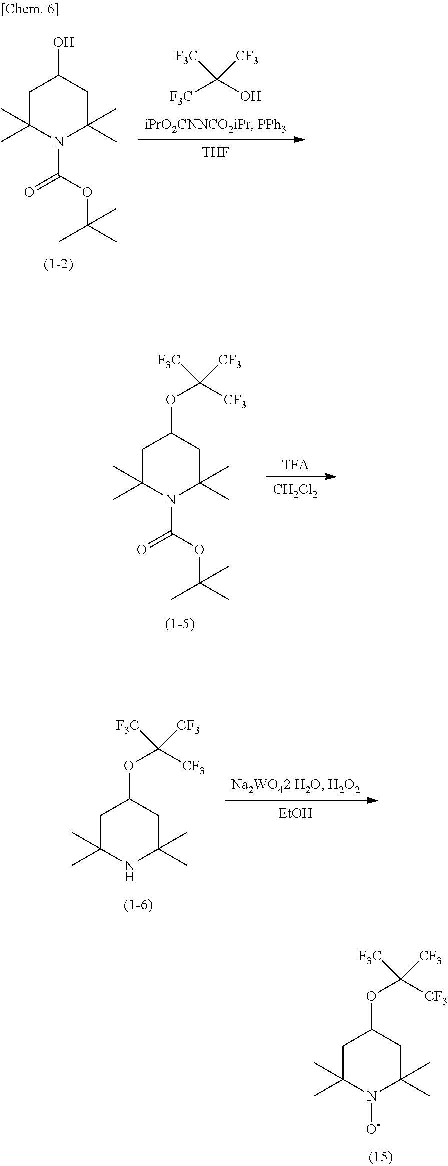

### Example 6

### Synthesis of Compound 16

A compound represented by Formula (16) which was a target product was synthesized in the same manner as in Example 5 except that 1,1,1,3,3,3-hexafluoro-2-propanol was used instead of nonafluoro-tert-butanol.

When the resulting compound was subjected to mass spectrometry, a peak was confirmed at m/z=322 (M⁺). From this, it was confirmed that the synthesized compound is a compound represented by Formula (16). In addition, the purity of the compound represented by Formula (16) which was conformed through high-performance liquid chromatography (HPLC) was 97.5%.

### Example 7

### Synthesis of Compound 17

A compound represented by Formula (17) which was a target product was synthesized in the same manner as in Example 5 except that 2,2,2-trifluoroethanol was used instead of nonafluoro-tert-butanol.

When the resulting compound was subjected to mass spectrometry, a peak was confirmed at m/z=254 (M⁺). From this, it was confirmed that the synthesized compound is a compound represented by Formula (17). In addition, the purity of the compound represented by Formula (17) which was conformed through high-performance liquid chromatography (HPLC) was 96.9%.

### Example 8

### Synthesis of Compound 18

A compound represented by Formula (18) which was a target product was synthesized in the same manner as in Example 5 except that 2-hydroxybenzotrifluoride was used instead of nonafluoro-tert-butanol.

When the resulting compound was subjected to mass spectrometry, a peak was confirmed at m/z=316 (M⁺). From this, it was confirmed that the synthesized compound is a compound represented by Formula (18). In addition, the purity of the compound represented by Formula (18) which was conformed through high-performance liquid chromatography (HPLC) was 95.2%.

### Example 9

### Synthesis of Compound 19

A compound represented by Formula (19) which was a target product was synthesized in the same manner as in Example 5 except that 3-hydroxybenzotrifluoride was used instead of nonafluoro-tert-butanol.

When the resulting compound was subjected to mass spectrometry, a peak was confirmed at m/z=316 (M⁺). From this, it was confirmed that the synthesized compound is a compound represented by Formula (19). In addition, the purity of the compound represented by Formula (19) which was conformed through high-performance liquid chromatography (HPLC) was 95.7%.

### Example 10

### Synthesis of Compound 20

A compound represented by Formula (20) which was a target product was synthesized in the same manner as in Example 5 except that 3,5-bis(trifluoro)phenol was used instead of nonafluoro-tert-butanol.

When the resulting compound was subjected to mass spectrometry, a peak was confirmed at m/z=384 (M⁺). From this, it was confirmed that the synthesized compound is a compound represented by Formula (20). In addition, the purity of the compound represented by Formula (20) which was conformed through high-performance liquid chromatography (HPLC) was 95.0%.

### Example 11

### Synthesis of Compound 21

A compound represented by Formula (21) which was a target product was synthesized in the same manner as in Example 5 except that 4-hydroxybenzotrifluoride was used instead of nonafluoro-tert-butanol.

When the resulting compound was subjected to mass spectrometry, a peak was confirmed at m/z=316 (M⁺). From this, it was confirmed that the synthesized compound is a compound represented by Formula (21). In addition, the purity of the compound represented by Formula (21) which was conformed through high-performance liquid chromatography (HPLC) was 97.0%.

### Example 12

### Synthesis of Compound 22

### Synthesis of 1,2,2,6,6-pentamethyl-4-piperidone (1-7)

In an argon stream, 15.524 g (100 mmol) of 2,2,6,6-tetramethylpiperidine-4-one, 23.288 g (150 mmol) of paraformaldehyde, and 1(X) mL of toluene were mixed with each other and heated to 90° C. 5.70 mL (150 mmol) of formic acid was added dropwise thereto over 30 minutes, and the mixture was heated at 100° C. for 12 hours to cause a reaction.

After the reaction solution was cooled to room temperature and 2.000 g (50 mmol) of sodium hydroxide was added thereto and stirred for 1 hour, suction filtration was performed, and the filtrate was concentrated under reduced pressure. The obtained concentrate was distilled under reduced pressure (70-72° C./2 mmHg) to obtain 1,2,2,6,6-pentamethyl-4-piperidone which was a target product and represented by Formula (1-7) (yield of 13.532 g, percent yield of 80%).

### Synthesis of 2,2,6,6-tetraethyl-4-piperidone (1-8)

In an argon stream, 13.532 g (80.0 mmol) of 1,2,2,6,6-pentamethyl-4-piperidone (1-7) synthesized through the above-described reaction and 25.3 mL (240 mmol) of 3-pentanone were dissolved in 100 mL of dimethyl sulfoxide (DMSO), and 25.675 g (480 mmol) of ammonium chloride was added thereto over 30 minutes.

After the reaction mixture was stirred at 60° C. for 5 hours and cooled to room temperature, water was added thereto, and the mixture was neutralized with IN hydrochloric acid. After extraction was performed with diethyl ether, the pH in the water tank was adjusted to 9 with a 10% potassium carbonate aqueous solution, and extraction was performed with ethyl acetate. After washing the organic layer with saturated saline, the washed organic layer was dried with magnesium sulfate and concentrated under reduced pressure. The resulting crude product was purified through silica gel column chromatography (hexane:ethyl acetate=9:1) to obtain 2,2,6,6-tetraethyl-4-piperidone which was a target product and represented by Formula (1-8) (yield of 6.758 g, percent yield of 40%).

### Synthesis of tert-butyl-2,2,6,6-tetraethyl-4-oxopiperidine-1-carboxylic acid ester (1-9)

6.758 g (32.0 mmol) of 2,2,6,6-tetraethyl-4-piperidone (1-8) synthesized through the above-described reaction was dissolved in 30 mL of tetrahydrofuran (THF) and cooled in an ice bath. 30 mL of a tetrahydrofuran solution of 9.34 mL (67.2 mmol) of triethylamine (Et₃N) and 4.713 g (33.6 mmol) of di-tert-butyldicarbonate (Bdoc₂O) was added thereto and stirred at room temperature for 2 hours to cause a reaction.

The reaction solution was concentrated under reduced pressure, and hexane was added thereto. After the resulting solid was filtered off, it was washed with hexane to obtain tert-butyl-2,2,6,6-tetraethyl-4-oxopiperidine-1-carboxylic acid ester (yield of 7.769 g. percent yield of 78%) represented by Formula (1-9) which was a target product in which a tertiary butoxycarbonyl group (t-Boc group) which was a protecting group was bound to a nitrogen atom of a piperidine ring.

### Synthesis of tert-butyl-4-hydroxy-2,2,6,6-tetraethyl-piperidine-1-carboxylic acid ester (1-10)

In an argon stream, 7.769 g (25.0 mmol) of tert-butyl-2,2,6,6-tetraethyl-4-oxopiperidine-1-carboxylic acid ester (1-9) synthesized through the above-described reaction was dissolved in 15 mL of ethanol (EtOH) and cooled in an ice bath. 0.495 g (13.0 mmol) of sodium borohydride was slowly added thereto and stirred at room temperature for 6 hours to cause a reaction.

Saturated saline was added to the reaction solution, extraction was performed with ethyl acetate, and the extract was dried with magnesium sulfate. The extract was concentrated under reduced pressure, and tert-butyl-4-hydroxy-2,2,6,6-tetraethyl-piperidine-1-carboxylic acid ester which was a target product and represented by Formula (1-10) was obtained (yield of 6.735 g, percent yield of 86%).

### Synthesis of Compound (1-11)

In an argon stream, 10 mL of tetrahydrofuran (THF) was added to 1.126 g (25.8 mmol) of 55% sodium hydride and cooled in an ice bath. 30 mL of a tetrahydrofuran solution of 6.735 g (21.5 mmol) of tert-butyl-4-hydroxy-2,2,6,6-tetraethyl-piperidine-1-carboxylic acid ester (1-10) synthesized through the above-described reaction was added thereto over 20 minutes and stirred for 30 minutes. 20 mL of a tetrahydrofuran solution of 9.572 g (25.8 mmol) of 1-bromo-4-((1,1,1,3,3,3-hexafluoro-2-(trifluoromethyl)propane-2-yl)oxy)butane was added thereto over 10 minutes and stirred at room temperature for 12 hours to cause a reaction.

Water was added to the reaction solution, extraction was performed with diethyl ether, and the extract was dried with magnesium sulfate. The extract was concentrated under reduced pressure, the resulting crude product was purified through silica gel column chromatography (hexane:ethyl acetate=9:1 to 4:1) to obtain a compound which was a target product and represented by Formula (1-11) (yield of 6.070 g, percent yield of 47%).

### Synthesis of 4-(4-((1,1,1,3,3,3-hexafluoro-2-(trifluoromethyl)propane-2-yl)oxy)butoxy)-2,2,6,6-tetraet hylpiperidine (1-12)

6.070 g (10.1 mmol) of the compound (1-11) synthesized through the above-described reaction was dissolved in 30 mL of dichloromethane. 5.37 mL (70 mmol) of trifluoroacetic acid (TFA) was added thereto and stirred at room temperature for 18 hours to cause a reaction, and a tertiary butoxycarbonyl group which was a protecting group was removed.

After the reaction solution was concentrated under reduced pressure, water was added thereto, and the organic layer was neutralized with a sodium hydrogen carbonate aqueous solution. After extraction was performed with diethyl ether, the organic layer was washed with saturated saline and dried with magnesium sulfate. The organic layer was concentrated under reduced pressure, and 4-(4-((1,1,1,3,3,3-hexafluoro-2-(trifluoromethyl)propane-2-yl)oxy)butoxy)-2,2,6,6-tetraet hylpiperidine which was a target product and represented by Formula (1-12) was obtained (yield of 4.574 g, percent yield of 90%).

### Synthesis of 4-(4-((1,1,1,3,3,3-hexafluoro-2-(trifluoromethyl)propane-2-yl)oxy)butoxy)-2,2,6,6-tetraet hylpiperidine-1-oxyl (22)

4.574 g (9.09 mmol) of 4-(4-((1,1,1,3,3,3-hexafluoro-2-(trifluoromethyl)propane-2-yl)oxy)butoxy)-2,2,6,6-tetraet hylpiperidine (1-12) synthesized through the above-described reaction, 0.330 g (1.00 mmol) of sodium tungstate dihydrate, and 5 mL of ethanol (EtOH) were mixed with each other and cooled in an ice bath. 10 mL (95.3 mmol) of a 30% hydrogen peroxide solution was slowly added thereto and stirred at room temperature for 24 hours. Potassium carbonate was added the reaction solution, extraction was performed with chloroform, and the extract was dried with magnesium sulfate.

After the extract was concentrated under reduced pressure, the resulting crude product was purified through silica gel column chromatography (hexane:ethyl acetate 9:1) to obtain 4-(4-((1,1,1,3,3,3-hexafluoro-2-(trifluoromethyl)propane-2-yl)oxy)butoxy)-2,2,6,6-tetraet hylpiperidine-1-oxyl which was a target product and represented by Formula (22) (yield of 2.968 g. percent yield of 63%).

When the resulting compound was subjected to mass spectrometry, a peak was confirmed at m/z=518 (M⁺). From this, it was confirmed that the synthesized compound is a compound represented by Formula (22). In addition, the purity of the compound represented by Formula (22) which was conformed through high-performance liquid chromatography (HPLC) was 95.4%.

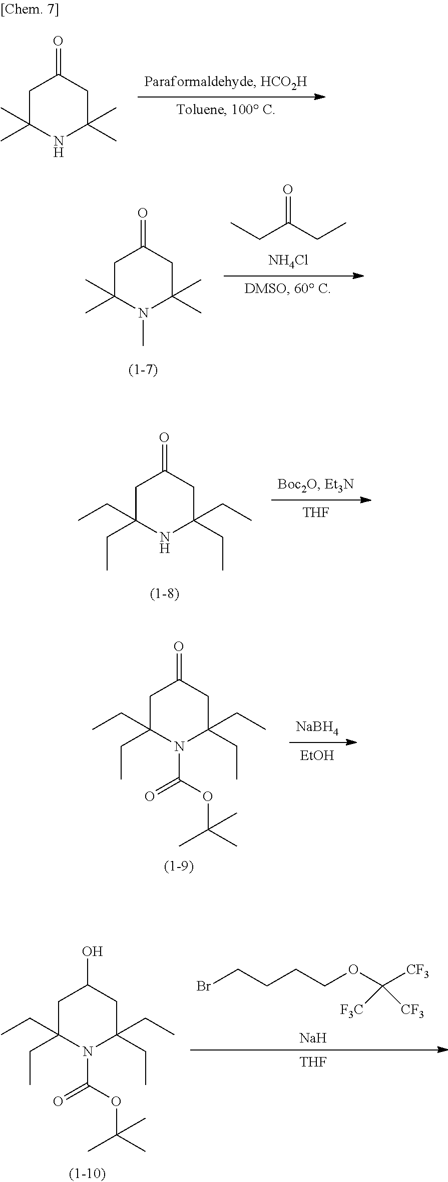

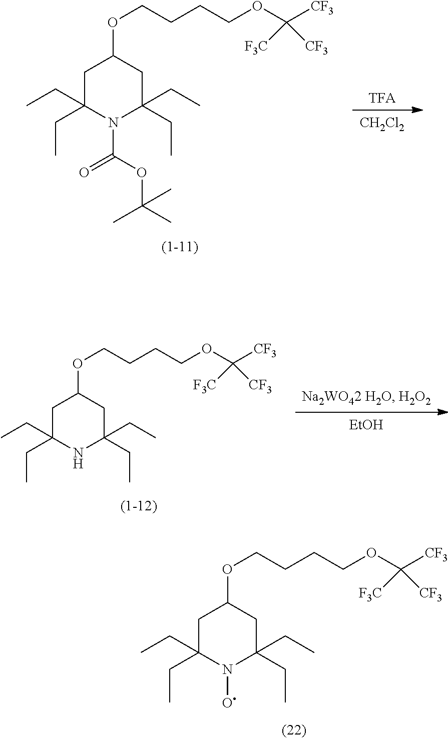

### Example 13

### Synthesis of Compound 23

A compound represented by Formula (23) which was a target product was synthesized in the same manner as in Example 12 except that 2-(2-bromoethoxy)-1,1,1,3,3,3-hexafluoro-2-(trifluoromethyl)propane was used instead of 1-bromo-4-((1,1,1,3,3,3-hexafluoro-2-(trifluoromethyl)propane-2-yl)oxy)butane.

When the resulting compound was subjected to mass spectrometry, a peak was confirmed at m/z=490 (M⁺). From this, it was confirmed that the synthesized compound is a compound represented by Formula (23). In addition, the purity of the compound represented by Formula (23) which was conformed through high-performance liquid chromatography (HPLC) was 96.6%.

### Example 14

### Synthesis of Compound 24

### Synthesis of Compound (1-13)

In an argon stream, 3.133 g (10.0 mmol) of tert-butyl-4-hydroxy-2,2,6,6-tetraethyl-piperidine-1-carboxylic acid ester (1-10) synthesized through the above-described reaction, 2.08 mL (15.0 mmol) of nonafluoro-tert-butanol, 3.934 g (15.0 mmol) of triphenylphosphine (PPh₃), and 40 mL of tetrahydrofuran (THF) were mixed with each other and cooled in an ice bath. 2.92 mL (15.0 mmol) of diisopropyl azodicarboxylate (iPrO₂CNNCO₂iPr) was added dropwise thereto over 10 minutes and stirred at room temperature for 24 hours to cause a reaction.

The reaction solution was concentrated under reduced pressure and purified through silica gel column chromatography (hexane:ethyl acetate=9:1 to 4:1) to obtain a compound which was a target product and represented by Formula (1-13) (yield of 2.327 g, percent yield of 45%).

### Synthesis of 4-((1,1,1,3,3,3-hexafluoro-2-(trifluoromethyl)propane-2-yl)oxy)-2,2,6,6-tetramethylpiperid ine (1-14)

2.327 g (4.50 mmol) of the compound (1-13) synthesized through the above-described reaction was dissolved in 15 mL of dichloromethane, 3.1 mL (40.0 mmol) of trifluoroacetic acid (TFA) was added thereto and stiffed at room temperature for 18 hours to cause a reaction, and a tertiary butoxycarbonyl group which was a protecting group was removed. After the reaction solution was concentrated under reduced pressure, water was added thereto, and the organic layer was neutralized with a sodium hydrogen carbonate aqueous solution. After extraction was performed with diethyl ether, the organic layer was washed with saturated saline and dried with magnesium sulfate.

The organic layer was concentrated under reduced pressure, and 4-((1,1,1,3,3,3-hexafluoro-2-(trifluoromethyl)propane-2-yl)oxy)-2,2,6,6-tetramethylpiperid ine which was a target product and represented by Formula (1-14) was obtained (yield of 1.746 g, percent yield of 90%).

### Synthesis of 4-((1,1,1,3,3,3-hexafluoro-2-(trifluoromethyl)propane-2-yl)oxy)-2,2,6,6-tetramethylpiperid ine-1-oxyl(22)

1.746 g (4.05 mmol) of 4-((1,1,1,3,3,3-hexafluoro-2-(trifluoromethyl)propane-2-yl)oxy)-2,2,6,6-tetramethylpiperid ine (1-14) synthesized through the above-described reaction, 0.132 g (0.40 mmol) of sodium tungstate dihydrate, and 5 mL of ethanol (EtOH) were mixed with each other and cooled in an ice bath. 15 mL (143 mmol) of a 30% hydrogen peroxide solution was slowly added thereto and stirred at room temperature for 24 hours to cause a reaction. Potassium carbonate was added the reaction solution, extraction was performed with chloroform, and the extract was dried with magnesium sulfate.

After the extract was concentrated under reduced pressure, the resulting crude product was purified through silica gel column chromatography (hexane:ethyl acetate=9:1) to obtain 4-((1,1,1,3,3,3-hexafluoro-2-(trifluoromethyl)propane-2-yl)oxy)-2,2,6,6-tetramethylpiperid ine-1-oxyl which was a target product and represented by Formula (22) (yield of 0.891 g, percent yield of 51%).

When the resulting compound was subjected to mass spectrometry, a peak was confirmed at m/z. 431 (M⁺). From this, it was confirmed that the synthesized compound is a compound represented by Formula (24). In addition, the purity of the compound represented by Formula (24) which was conformed through high-performance liquid chromatography (HPLC) was 96.2%.

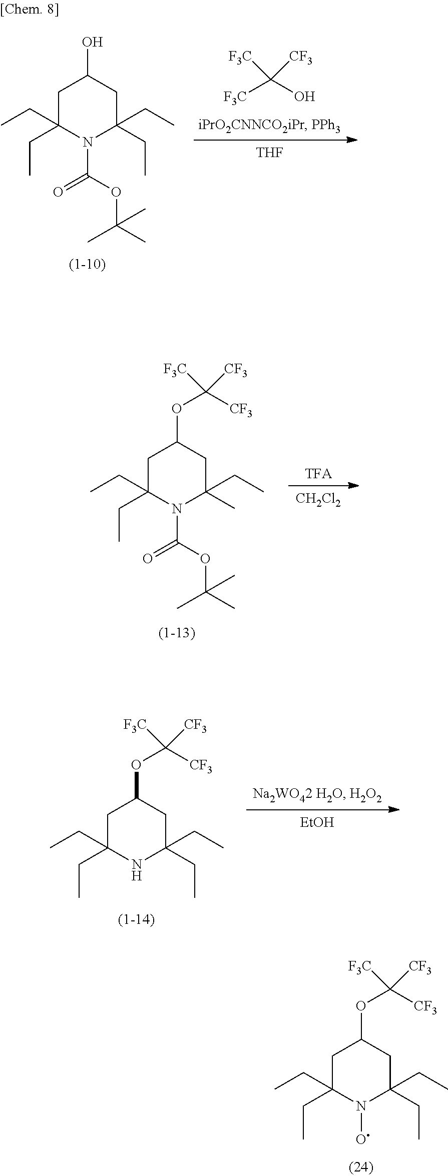

### Example 15

### Synthesis of Compound 25

### Synthesis of 2,2-diethyl-6,6-dimethyl-4-piperidone (1-15)

In an argon stream, 13.532 g (40.0 mmol) of 1,2,2,6,6-pentamethyl-4-piperidone (1-7) synthesized in the same manner as in Example 12 and 25.3 mL (60.0 mmol) of 3-pentanone were dissolved in 50 mL of dimethyl sulfoxide (DMSO), and 25.675 g (240 mmol) of ammonium chloride was added thereto over 30 minutes. After the reaction mixture was stirred at 60° C. for 5 hours and cooled to room temperature, water was added thereto, and the mixture was neutralized with IN hydrochloric acid. After extraction was performed with diethyl ether, the pH in the water tank was adjusted to 9 with a 10% potassium carbonate aqueous solution, and extraction was performed with ethyl acetate.

After washing the organic layer with saturated saline, the washed organic layer was dried with magnesium sulfate and concentrated under reduced pressure. The resulting crude product was purified through silica gel column chromatography (hexane:ethyl acetate=9:1) to obtain 2,2-diethyl-6,6-dimethyl-4-piperidone which was a target product and represented by Formula (1-15) (yield of 1.100 g, percent yield of 15%).

### Synthesis of tert-butyl-2,2-diethyl-6,6-dimethyl-4-oxopiperidine-1-carboxylic acid ester (1-16)

1.1(0 g (6.00 mmol) of 2,2-diethyl-6,6-dimethyl-4-piperidone (1-15) synthesized through the above-described reaction was dissolved in 10 mL of tetrahydrofuran (THF) and cooled in an ice bath. 10 mL of a tetrahydrofuran solution of 1.76 mL (12.6 mmol) of triethylamine (Et₃N) and 1.440 g (6.60 mmol) of di-tert-butyldicarbonate (Boc₂O) was added thereto and stirred at room temperature for 2 hours to cause a reaction.

The reaction solution was concentrated under reduced pressure, and hexane was added thereto. After the resulting solid was filtered off, it was washed with hexane to obtain tert-butyl-2,2-diethyl-6,6-dimethyl-4-oxopiperidine-1-carboxylic acid ester (yield of 1.394 g, percent yield of 82%) represented by Formula (1-16) which was a target product in which a tertiary butoxycarbonyl group (t-Boc group) which was a protecting group was bound to a nitrogen atom of a piperidine ring.

### Synthesis of tert-butyl-4-hydroxy-2,2-diethyl-6,6-dimethylpiperidine-1-carboxylic acid ester (1-17)

In an argon stream, 1.394 g (4.92 mmol) of tert-butyl-2,2-diethyl-6,6-dimethyl-4-oxopiperidine-1-carboxylic acid ester (1-16) synthesized through the above-described reaction was dissolved in 5 mL of ethanol (EtOH) and cooled in an ice bath. 0.095 g (2.50 mmol) of sodium borohydride was slowly added thereto and stirred at room temperature for 6 hours to cause a reaction.

Saturated saline was added to the reaction solution, extraction was performed with ethyl acetate, and the extract was dried with magnesium sulfate. The extract was concentrated under reduced pressure, and tert-butyl-4-hydroxy-2,2-diethyl-6,6-dimethylpiperidine-1-carboxylic acid ester which was a target product and represented by Formula (1-17) was obtained (yield of 1.208 g, percent yield of 86%).

### Synthesis of Compound (1-18)

In an argon stream, 5 mL of tetrahydrofuran (THF) was added to 0.222 g (5.08 mmol) of 55% sodium hydride and cooled in an ice bath. 5 mL of a tetrahydrofuran solution of 1.208 g (4.23 mmol) of tert-butyl-4-hydroxy-2,2-diethyl-6,6-dimethylpiperidine-1-carboxylic acid ester (1-17) synthesized through the above-described reaction was added thereto over 20 minutes and stirred for 30 minutes.

5 mL of a tetrahydrofuran solution of 1.885 g (5.08 mmol) of 1-bromo-4-((1,1,1,3,3,3-hexafluoro-2-(trifluoromethyl)propane-2-yl)oxy)butane was added thereto over 10 minutes and stirred at room temperature for 12 hours to cause a reaction.

Water was added to the reaction solution, extraction was performed with diethyl ether, and the extract was dried with magnesium sulfate. The extract was concentrated under reduced pressure, the resulting crude product was purified through silica gel column chromatography (hexane:ethyl acetate=9:1 to 4:1) to obtain a compound which was a target product and represented by Formula (1-18) (yield of 1.217 g, percent yield of 50%).

### Synthesis of 4-(4-((1,1,1,3,3,3-hexafluoro-2-(trifluoromethyl)propane-2-yl)oxy)butoxy)-2,2-diethyl-6,6-dimethylpiperidine (1-19)

1.217 g (2.12 mmol) of the compound (1-18) synthesized through the above-described reaction was dissolved in 5 mL of dichloromethane, 1.1 mL (14.0 mmol) of trifluoroacetic acid (TFA) was added thereto and stirred at room temperature for 18 hours to cause a reaction, and a tertiary butoxycarbonyl group which was a protecting group was removed.

After the reaction solution was concentrated under reduced pressure, water was added thereto, and the organic layer was neutralized with a sodium hydrogen carbonate aqueous solution. After extraction was performed with diethyl ether, the organic layer was washed with saturated saline and dried with magnesium sulfate. The organic layer was concentrated under reduced pressure, and 4-(4-((1,1,1,3,3,3-hexafluoro-2-(trifluoromethyl)propane-2-yl)oxy)butoxy)-2,2-diethyl-6,6-dimethylpiperidine which was a target product and represented by Formula (1-19) was obtained (yield of 0.907 g, percent yield of 90%).

### Synthesis of 4-(4-((1,1,1,3,3,3-hexafluoro-2-(trifluoromethyl)propane-2-yl)oxy)butoxy)-2,2-diethyl-6,6-dimethylpiperidine-1-oxyl (25)

0.907 g (1.91 mmol) of 4-(4-((1,1,1,3,3,3-hexafluoro-2-(trifluoromethyl)propane-2-yl)oxy)butoxy)-2,2-diethyl-6,6-dimethylpiperidine (1-19) synthesized through the above-described reaction, 0.066 g (0.200 mmol) of sodium tungstate dihydrate, and 5 mL of ethanol (EtOH) were mixed with each other and cooled in an ice bath. 2 mL (19 mmol) of a 30% hydrogen peroxide solution was slowly added thereto and stirred at room temperature for 24 hours to cause a reaction. Potassium carbonate was added the reaction solution, extraction was performed with chloroform, and the extract was dried with magnesium sulfate.

After the extract was concentrated under reduced pressure, the resulting crude product was purified through silica gel column chromatography (hexane:ethyl acetate=9:1) to obtain 4-(4-((1,1,1,3,3,3-hexafluoro-2-(trifluoromethyl)propane-2-yl)oxy)butoxy)-2,2-diethyl-6, 6-dimethylpiperidine-1-oxyl which was a target product and represented by Formula (25) (yield of 0.562 g, percent yield of 60%).

When the resulting compound was subjected to mass spectrometry, a peak was confirmed at m/z=490 (M⁺). From this, it was confirmed that the synthesized compound is a compound represented by Formula (25). In addition, the purity of the compound represented by Formula (25) which was conformed through high-performance liquid chromatography (HPLC) was 95.0%.

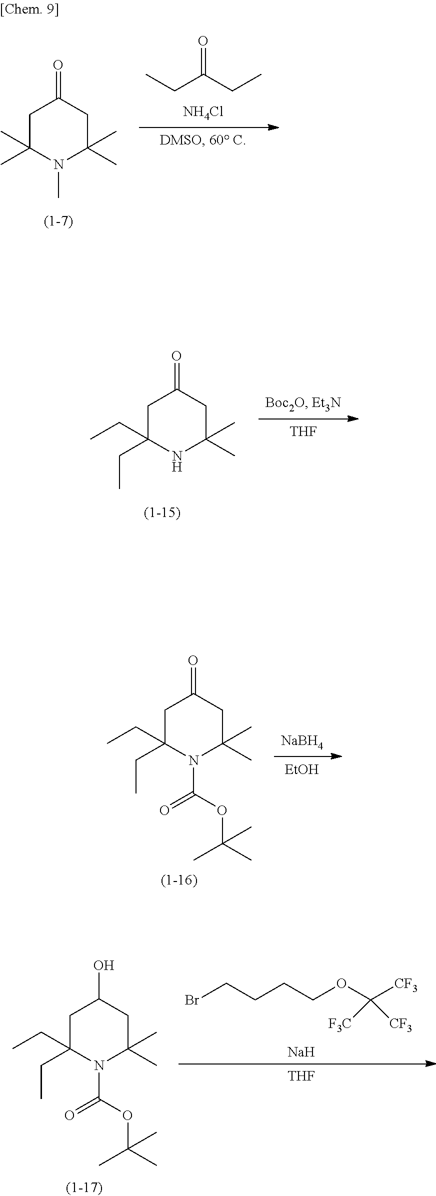

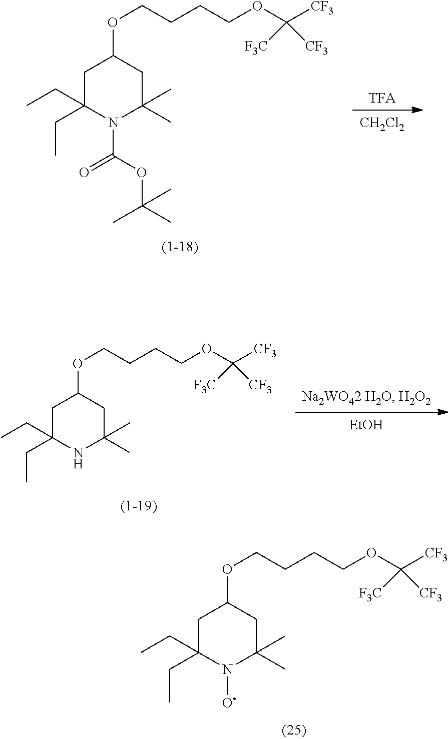

### Example 16

### Synthesis of Compound 26

### Synthesis of 4-(4-((1,1,1,3,3,3-hexafluoropropane-2-yl)oxy)butoxy)-2,2,6,6-tetramethylpiperidine-1-o xyl 1(26)

A compound represented by Formula (26) which was a target product was synthesized in the same manner as in Example 1 except that 1-bromo-4-(1,1,1,3,3,3-hexafluoropropane-2-yl)oxy)butane was used instead of 1-bromo-4-((1,1,1,3,3,3-hexafluoro-2-(trifluoromethyl)propane-2-yl)oxy)butane.

When the resulting compound was subjected to mass spectrometry, a peak was confirmed at m/z=394 (M⁺). From this, it was confirmed that the synthesized compound is a compound represented by Formula (26). In addition, the purity of the compound represented by Formula (26) which was conformed through high-performance liquid chromatography (HPLC) was 96.5%.

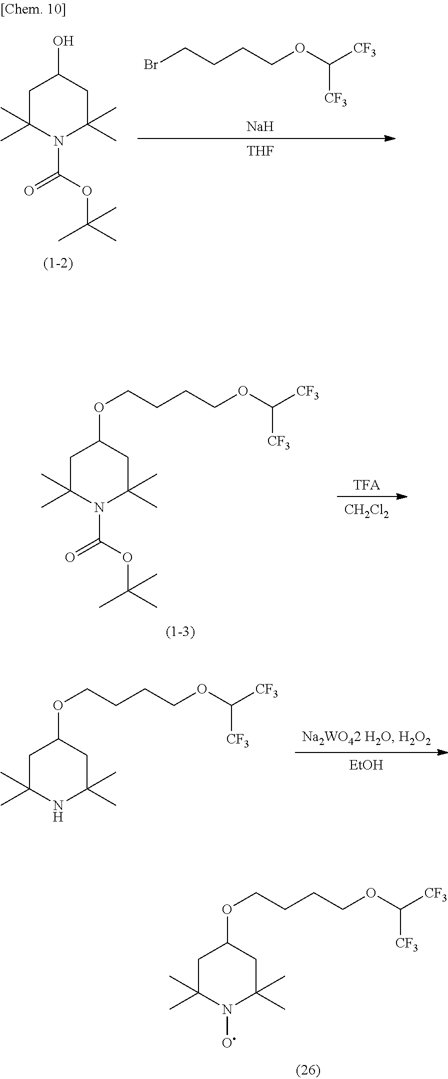

### Example 17

### Synthesis of Compound 27

### Synthesis of 4-((8-bromooctyl)oxy)-2,2,6,6-tetramethylpiperidine-1-oxyl (1-20)

In an argon stream, 15 mL of dimethylformamide (DMF) was added to 1.047 g (24.0 mmol) of 55% sodium hydride (NaH), and the mixture was stirred at room temperature for 10 minutes. 30 mL of a dimethylformamide solution containing 3.445 g (20.0 mmol) of 4-hydroxy-2,2,6,6-tetramethylpiperidine-1-oxyl was added dropwise thereto over 10 minutes, and the mixture was stiffed at room temperature for 3 hours. 5.55 mL (30.0 mmol) of 1,8-dibromooctane was added to the stinted solution in an ice bath and stiffed at room temperature for 20 hours to cause a reaction. Water was added to the reaction solution and extraction was performed with diethyl ether, and then the extract was washed with water, and the organic layer was dried with magnesium sulfate. The organic layer was concentrated under reduced pressure and then purified through silica gel column chromatography (hexane:ethyl acetate=9:1) to obtain 4-((8-bromooctyl)oxy)-2,2,6,6-tetramethylpiperidine-1-oxyl (1-20) which was a target product (yield of 2.091 g, percent yield of 29%).

### Synthesis of 4-((8-((1,1,1,3,3,3-hexafluoro-2-(trifluoromethyl)propane-2-yl)oxy)octyl)oxy)-2,2,6,6-tet ramethylpiperidine-1-oxyl (27)

In an argon stream, 2.091 g (5.75 mmol) of 4-((8-bromooctyl)oxy)-2,2,6,6-tetramethylpiperidine-1-oxyl (1-20) obtained through the above-described reaction and 2.225 g (8.63 mmol) of sodium-1,1,1,3,3,3-hexafluoro-2-(trifluoromethyl)-2-propanolate were dissolved in 10 mL of dimethylformamide and stirred at room temperature for 16 hours and then at 65° C. for 9 hours to cause a reaction. Water was added to the reaction solution and extraction was performed with diethyl ether, and then the extract was washed with water, and the organic laver was dried with magnesium sulfate. After the organic laver was concentrated under reduced pressure, it was purified through silica gel column chromatography (hexane:ethyl acetate=95:5) to obtain 4-((8-((1,1,1,3,3,3-hexafluoro-2-(trifluoromethyl)propane-2-yl)oxy)octyl)oxy)-2,2,6,6-tet ramethylpiperidine-1-oxyl (27) which was a target product (yield of 2.391 g, percent yield of 80%).

When the resulting compound was subjected to mass spectrometry, a peak was confirmed at m/z=519 (M⁺H). From this, it was confirmed that the synthesized compound is a compound represented by Formula (27). In addition, the purity of the compound represented by Formula (27) which was conformed through high-performance liquid chromatography (HPLC) was 95.9%.

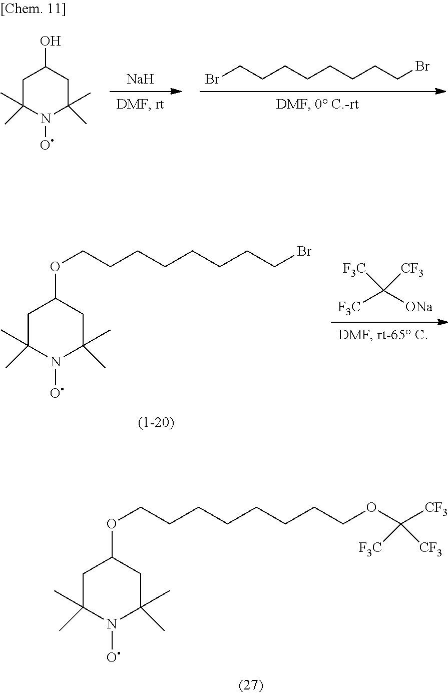

### Example 18

### Synthesis of Compound 28

A compound represented by Formula (28) which was a target product was synthesized in the same manner as in Example 17 except that 1,12-dibromododecane was used instead of 1,8-dibromooctane.

When the resulting compound was subjected to mass spectrometry, a peak was confirmed at m/z=574 (M⁺). From this, it was confirmed that the synthesized compound is a compound represented by Formula (28). In addition, the purity of the compound represented by Formula (28) which was conformed through high-performance liquid chromatography (HPLC) was 93.5%.

### Example 19

### Synthesis of Compound 29

A compound represented by Formula (29) which was a target product was synthesized in the same manner as in Example 17 except that 1,16-dibromooctadecane was used instead of 1.8-dibromooctane.

When the resulting compound was subjected to mass spectrometry, a peak was confirmed at m/z=630 (M⁺). From this, it was confirmed that the synthesized compound is a compound represented by Formula (29). In addition, the purity of the compound represented by Formula (29) which was conformed through high-performance liquid chromatography (HPLC) was 95.0%.

### Comparative Example 1

Trifluoromethylbenzene represented by Formula (A1) below was prepared.

### Comparative Example 2

### Synthesis of Compound A2

Trifluoromethylbenzene represented by Formula (A1) and 4-hydroxy-2,2,6,6-tetramethylpiperidine-1-oxyl free radical represented by Formula (A2) below (TEMPOL, manufactured by Tokyo Chemical Industry Co., Ltd.) were mixed with each other at a molar ratio ((A1):(A2)) of 1:1 to obtain a compound of Comparative Example 2.

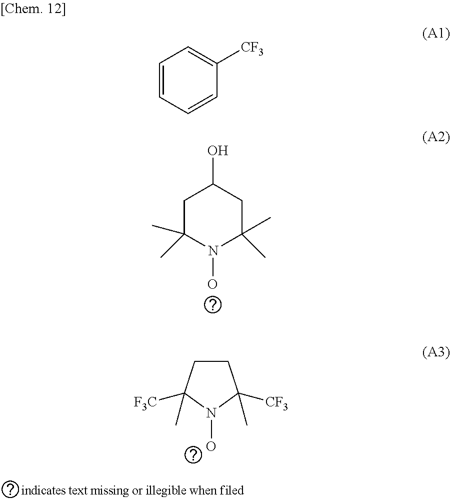

For the compounds of Examples 1 to 19 and Comparative Examples 1 and 2 thus obtained, the ¹⁹F spin-lattice relaxation time (T1) was measured through methods shown below. The results are shown in Table 1.

### (Measurement of ¹⁹F Spin-Lattice Relaxation Time (T1))

A compound was dissolved in a deuterated chloroform solution at a concentration of 50 mM, and the longitudinal relaxation time (T1) of ¹⁹F nucleus was measured through an inversion recovery method using a 500 MHz NMR device under the conditions shown below.

Measurement Conditions
NMR device: JNM-ECA500 (manufactured by JEOL)
Measurement temperature: 36° C.
Pulse sequence: double_pulse
relaxation_delay: 10[s]
tau_interval: 4, 3, 2, 1, 0.8, 0.6, 0.4, 0.2, 0.1[s], 80, 60, 40, 20, 10, 8, 6, 4, 2[ms]
Number of times of integration: 16 times

In addition, for the compounds of Examples 1 to 19 and the compound of Comparative Example 3 represented by Formula (A3) above, the energy level of each semi-occupied molecular orbital (SOMO) was calculated through the method shown below. The results are shown in Table 1.

### (Calculation of Energy Level of SOMO)

Gaussian 09 manufactured by Gaussian, Inc. was used to calculate molecular orbitals of the compounds. The energy level of each semi-occupied molecular orbital (SOMO) was calculated through a structure optimization calculation by density-functional theory (DFT) in which B3LYP was used as a functional and 6-31+G(d,p) was used as a basis function.

TABLE 1

| | | T1 | SOMO |
|---|---|---|---|
| | Compound | 50 mM/CDCl₃ (sec) | (eV) |
| Example 1 | 11 | 0.134 | −2.385 |
| Example 2 | 12 | 0.121 | −2.430 |
| Example 3 | 13 | 0.105 | −2.419 |
| Example 4 | 14 | 0.117 | −2.432 |
| Example 5 | 15 | 0.094 | −2.513 |
| Example 6 | 16 | 0.096 | −2.519 |
| Example 7 | 17 | 0.101 | −2.466 |
| Example 8 | 18 | 0.098 | −2.464 |
| Example 9 | 19 | 0.110 | −2.422 |
| Example 10 | 20 | 0.108 | −2.558 |
| Example 11 | 21 | 0.123 | −2.422 |
| Example 12 | 22 | 0.130 | −2.313 |
| Example 13 | 23 | 0.118 | −2.313 |
| Example 14 | 24 | 0.092 | −2.395 |
| Example 15 | 25 | 0.127 | −2.367 |
| Example 16 | 26 | 0.130 | −2.367 |
| Example 17 | 27 | 0.130 | −2.395 |
| Example 18 | 28 | 0.214 | −2.395 |
| Example 19 | 29 | 0.284 | −2.395 |
| Comparative Example 1 | A1 | 0.353 | — |
| Comparative Example 2 | A1, A2 | 0.440 | — |
| Comparative Example 3 | A3 | — | −2.395 |

As shown in Table 1, the compounds of Examples 1 to 19 had shorter ¹⁹F spin-lattice relaxation time (T1) than the compounds of Comparative Examples 1 and 2.

In addition, the compounds of Examples 1 to 19 had higher energy levels of semi-occupied molecular orbitals (SOMO) than the compound of Comparative Example 3.

This is because Comparative Example 3 (compound A3) has only one carbon atom between a fluorine atom and carbon atoms at the 2- and 5-positions of a pyrrolidine ring and has a shorter distance between the fluorine atom and a nitroxide radical than the compounds of Examples 1 to 19. As a result, the nitroxide radical contained in Comparative Example 3 (compound A3) is susceptible to electronic influence from the fluorine atom, and therefore it is inferred that the SOMO energy level would be lowered due to the effect of the fluorine atom as an electron withdrawing group.

In addition, for each of the compounds of Example 1 and Comparative Example 1, a 5 mM deuterated chloroform solution and a 10 mM deuterated chloroform solution were adjusted, and T1-weighted images (phantom images) were obtained under the following imaging conditions.

(Imaging conditions)
Imaging device: MRI BioSpec117/11 (manufactured by Burker)
Pulse sequence: RAREVTR
Repetition time: TR=1500 ms
Echo time: TE=12 ms
Number of times of integration: 36 times
Total imaging time: 16 minutes 33 seconds

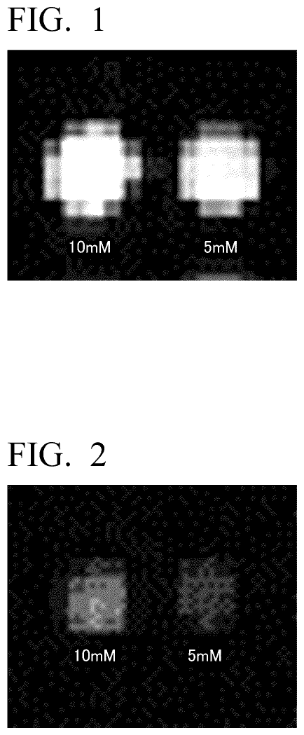

FIG. 1 is a T1-weighted ¹⁹F-MRI image of Example 1 (compound 11). FIG. 2 is a T1-weighted ¹⁹F-MRI image of Comparative Example 1 (compound A1).

The image of Example 1 (compound 1) shown in FIG. 1 was brighter than the image of Comparative Example 1 (compound A1) shown in FIG. 2 in either case of the 5 mM deuterated chloroform solution or the 10 mM deuterated chloroform solution.

In addition, it was possible to confirm from FIG. 1 that sufficiently clinically applicable high-sensitivity images are obtained using Example 1 (compound 1) in a contrast medium for MM diagnosis using fluorine as a detection nucleus.

### INDUSTRIAL APPLICABILITY

A contrast medium which is highly stable in vivo can be provided. In addition, a high-sensitivity magnetic resonance image can be obtained.

1. A fluorine-containing compound represented by General Formula (1) below, 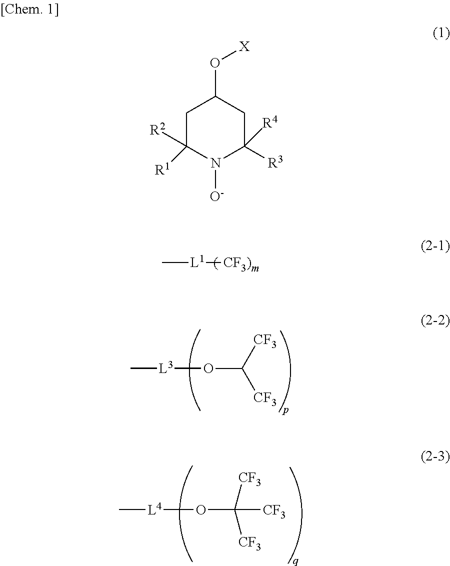
General Formula (1), R¹, R², R³, and R⁴ each independently represent a C1-10 alkyl group unsubstituted or substituted with a substituent containing no fluorine atoms, and X is a substituent represented by any of General Formulae (2-1), (2-2), and (2-3)),
(in General Formula (2-1), L¹ is either a C1-16 chain hydrocarbon group unsubstituted or substituted with a substituent containing no fluorine atoms or a linking group containing a C6-12 aryl group unsubstituted or substituted with a substituent containing no fluorine atoms, and m is an integer of 1 to 5),
(in General Formula (2-2), L³ is either a C1-16 chain hydrocarbon group unsubstituted or substituted with a substituent containing no fluorine atoms or a linking group containing a C6-12 aryl group unsubstituted or substituted with a substituent containing no fluorine atoms, and p is an integer of 1 to 5), and
(in General Formula (2-3), L⁴ is either a C1-16 chain hydrocarbon group unsubstituted or substituted with a substituent containing no fluorine atoms or a linking group containing a C6-12 aryl group unsubstituted or substituted with a substituent containing no fluorine atoms, and q is an integer of 1 to 5).

2. The fluorine-containing compound according to claim 1,
wherein R¹, R², R³, and R⁴ in General Formula (1) above each independently represent a C1-5 alkyl group unsubstituted or substituted with a substituent containing no fluorine atoms.

3. The fluorine-containing compound according to claim 1,
wherein L¹ in General Formula (2-1), L³ in General Formula (2-2), and L⁴ in General Formula (2-3) are C1-10 chain hydrocarbon groups unsubstituted or substituted with a substituent containing no fluorine atoms.

4. The fluorine-containing compound according to claim 1,
wherein L¹ in General Formula (2-1), L³ in General Formula (2-2), and L⁴ in General Formula (2-3) are linking groups containing a phenyl group.

5. The fluorine-containing compound according to claim 1,
wherein m in General Formula (2-1) is an integer of 1 to 3, and p in General Formula (2-2) and q in General Formula (2-3) are each 1 or 2.

6. The fluorine-containing compound according to claim 1, which is used in a contrast medium for magnetic resonance image diagnosis using fluorine as a detection nucleus.

7. A contrast medium for magnetic resonance image diagnosis using fluorine as a detection nucleus, comprising:
the fluorine-containing compound according to claim 1.
